(19) United States
(12) Patent Application Publication
(10) Pub. No.: US 2008/0154174 A1
(43) Pub. Date: Jun. 26, 2008

(54) ABSORBENT TAMPON HAVING OUTER PETALS

(76) Inventor: ROGERIO COSTA, Lorena (BR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/025,447

(22) Filed: Feb. 4, 2008

Related U.S. Application Data
(62) Division of application No. 10/167,672, filed on Jun. 11, 2002.

(51) Int. Cl. A61F 13/20; D04H 1/22

(52) U.S. Cl. 604/11; 604/379; 604/385.17; 28/118; 28/120

(57) ABSTRACT

A novel absorbent tampon suitable for insertion into a body cavity has a substantially elongate, compressed primary absorbent structure having a first end, a second end disposed opposite thereof, and a density of at least about 0.3 g/cm³. The tampon also includes a secondary absorbent structure secured to and extending from the first end of the primary absorbent structure and extending toward the second end of the primary absorbent structure. Preferably, the secondary absorbent structure is disposed about the primary absorbent structure and forms the outer circumferential surface of the tampon. The secondary absorbent structure is capable of extending outwardly from the primary absorbent structure upon the absorption of fluid into the tampon to increase the cross-sectional area of the tampon in the body cavity. In a preferred embodiment, the tampon has sufficient column strength for digital insertion (insertion without an applicator) into a vaginal canal.

### FIELD OF THE INVENTION

The present invention relates to an absorbent tampon suitable for insertion into a body cavity that is capable of relatively rapidly expanding and opening to form a structure with a relatively large cross-sectional area.

### BACKGROUND OF THE INVENTION

Absorbent tampons for insertion into body cavities are often cylindrically compressed fibrous products that expand relatively uniformly along their length. These tampons may expand into a generally cylindrical product as shown in Leutwyler et al., U.S. Pat. No. 5,813,102, or having a somewhat more square or flattened cross-section resulting from, e.g., Wolff et al., U.S. Pat. No. 3,422,496, or commercially available applicator tampons from Tambrands, Inc., under the Tampax® brand. These shapes do not necessarily match the cross-sectional shape of the body cavity, e.g., the vagina, into which they are placed, and may provide passages for early leakage of the absorbent product.

Other tampons have been designed to open into a cup-shaped form, such as described in Glassman, U.S. Pat. No. 3,618,605. This patent purports to describe a tampon having a wad or mass of highly absorbent material, preferably compressed, and additional absorbent layers that are drawn upwardly to enclose the central mass. When inserted into the vaginal passage for use, the entry end of the tampon purportedly expands to substantially fill the vaginal passage. However, this device is only described in use with an applicator to deliver the tampon into the vaginal passage. Thus, a the tampon appears to lack appropriate dimensional stability to be inserted without an applicator.

More recently, attempts have been made to design laterally expandable tampons as shown in Jessup et al., U.S. Pat. No. 6,039,716. This patent purports to disclose a tampon having an M-shaped profile that expands laterally after expulsion from an applicator during use.

Therefore, what is needed is a compressed tampon that quickly expands to fill a body cavity to prevent early leakage of bodily fluids therethrough. What is also needed is a tampon that rapidly expands and is capable of insertion without an applicator. What is also needed is a process that can provide such tampons.

### SUMMARY OF THE INVENTION

It is an object of the present invention to provide a compressed tampon that quickly expands to fill a body cavity to prevent early leakage of bodily fluids therethrough.

It is another object of the present invention to provide a tampon that rapidly expands and is capable of insertion without an applicator.

It is another object of the present invention to s provide a process that can provide such tampons.

In accordance with the present invention, there has been provided a novel absorbent tampon suitable for insertion into a body cavity. The tampon has a substantially elongate, compressed primary absorbent structure having a first end, a second end disposed opposite thereof, and a density of at least about 0.3 g/cm³. The tampon also includes a secondary absorbent structure secured to and extending from the first end of the primary absorbent structure and extending toward the second end of the primary absorbent structure. Preferably, the secondary absorbent structure is disposed about the primary absorbent structure and forms the outer circumferential surface of the tampon. The secondary absorbent structure is capable of extending outwardly from the primary absorbent structure to increase the cross-sectional area of the tampon in the body cavity. This is generally caused by the absorption of fluid into the tampon. In one preferred embodiment, the second end of the primary absorbent structure is disposed toward the insertion end of the tampon. In a preferred embodiment, the tampon has sufficient column strength for digital insertion (insertion without an applicator) into a vaginal canal.

In accordance with another aspect of the present invention, there has been provided a novel process of making an absorbent tampon. The process includes forming a substantially cylindrical, radially expanding primary absorbent structure having a first end and a second end disposed opposite thereof; securing an attachment zone of a secondary absorbent structure to the first end of the primary absorbent structure to form a tampon assembly; directing at least one distal portion of the secondary absorbent structure toward the first end of the primary absorbent structure, wherein the at least one distal portion is located at an edge of the secondary absorbent structure and spaced distal the attachment zone; and compressing the tampon assembly to form the absorbent tampon. The resulting tampon has a column strength of at least about 10 N.

### BRIEF DESCRIPTION OF THE DRAWING

FIG. 1 is a perspective view of a compressed tampon according to one embodiment of the present invention.

FIG. 2 is a perspective view of the tampon of FIG. 1 after expansion due to absorbed liquids.

FIG. 3 is a perspective view of a compressed tampon according to another embodiment of the present invention.

FIG. 4 is a perspective view of the tampon of FIG. 3 after expansion due to absorbed liquids.

FIGS. 5 and 6 are perspective views, similar to FIG. 2, of additional embodiments of the present invention.

FIG. 7 is a diagrammatic view of elements of an apparatus and a process for producing the tampon of FIG. 1.

### DETAILED DESCRIPTION OF THE PREFERRED EMBODIMENTS

As used in the specification and claims, the term “resilient” and variations of this term relate to materials that are capable of springing back into shape after a restraint is removed. An example is the release of restraint based upon over-compression of a fibrous structure, such as by absorption of liquids.

As used in the specification and claims, the term “compressible” and variations of this term relate to the ability to be compressed to hold a generally compressed form, but they also can expand to a relatively uncompressed state upon exposure to sufficient moisture.

As used in the specification and claims, the term “radially expand” and variations of this term relate to the expansion of elongate tampons. These tampons expand primarily in a direction perpendicular to the central, longitudinal axis of the tampon.

Absorbent tampons 10 are elongated, usually substantially cylindrical, masses of compressed absorbent material having a longitudinal axis “A” and a radius “R” that defines the outer circumferential surface 12 of the tampon 10. The elongated tampons 10 also have an insertion end 14 arranged and configured to ease insertion of the tampon 10 into a body cavity and a withdrawal end 16, opposite thereof. Tampons are often formed by first obtaining a shaped mass of absorbent material called a tampon blank. This blank can be in the form of a roll of sheet-like material, a segment of a continuous absorbent material, a mass of randomly or substantially uniformly oriented absorbent material, an individually prepared or cast mass of absorbent material, and the like. The blank can be formed of one absorbent unit, or it may be a combination of individual units or segments combined together.

The tampon blank is relatively uncompressed and has a relatively low density. It is then compressed in one or more steps to form a product having overall dimensions less than those of the blank. Tampons also usually include a cover 18 or some other surface treatment to contain the absorbent components and a withdrawal string 19 or other removal mechanism. The tampons are generally packaged in their compressed form for storage, shipment, and sale. Thus, the tampons, prior to use, are compact and discrete.

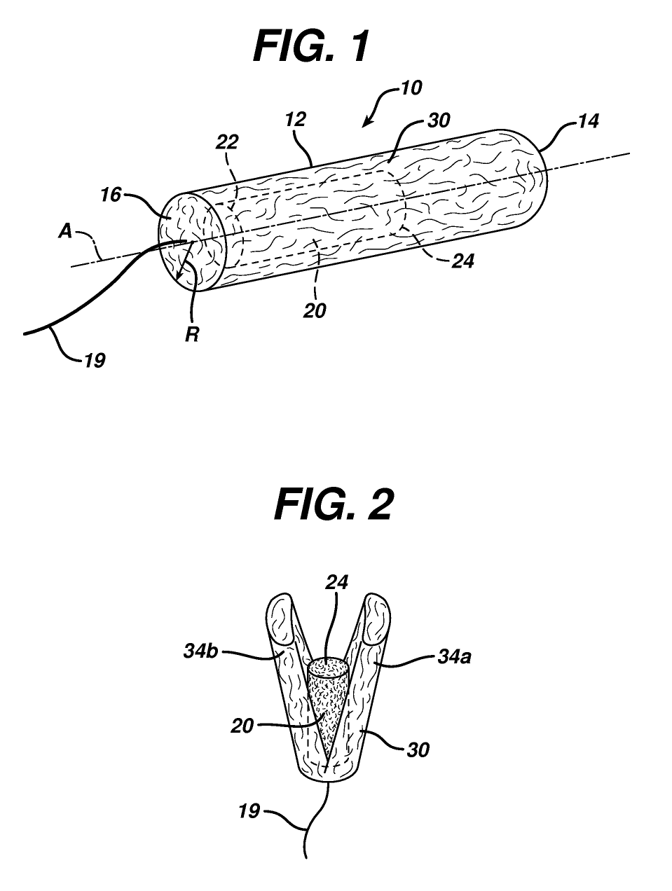

FIG. 1 illustrates one embodiment of the tampon 10 of the present invention. This tampon 10 is formed of a central, compressed elongate primary absorbent structure 20 substantially surrounding the longitudinal axis “A ” of the tampon 10 and a secondary absorbent structure 30 disposed about the primary absorbent structure 20 and forming the outer circumferential surface 18 of the tampon. The primary absorbent structure 20 is compressed to allow it to expand radially upon absorbing moisture and/or liquid upon absorbing sufficient liquid to cause it to swell or to otherwise release its resilient material from constraint in a compressed condition. The secondary absorbent structure 16 extends from an attachment zone 32 toward the second end 24 of the primary absorbent structure 20. In a preferred embodiment during use, the tampon 10 is inserted into a body cavity with the second end 22 directed toward a source of bodily fluid discharges. In use, the tampon 10 is inserted into a body cavity with the second end 24, corresponding to the insertion end 14 of the tampon 10, directed toward a source of bodily fluid discharges.

Early fluid insults of the tampon 10 should result in the primary absorbent structure absorbing 20 sufficient fluid to expand radially, forcing the secondary absorbent structure 30 to extend outwardly to increase the cross-sectional area of the tampon 10, as shown in FIG. 2.

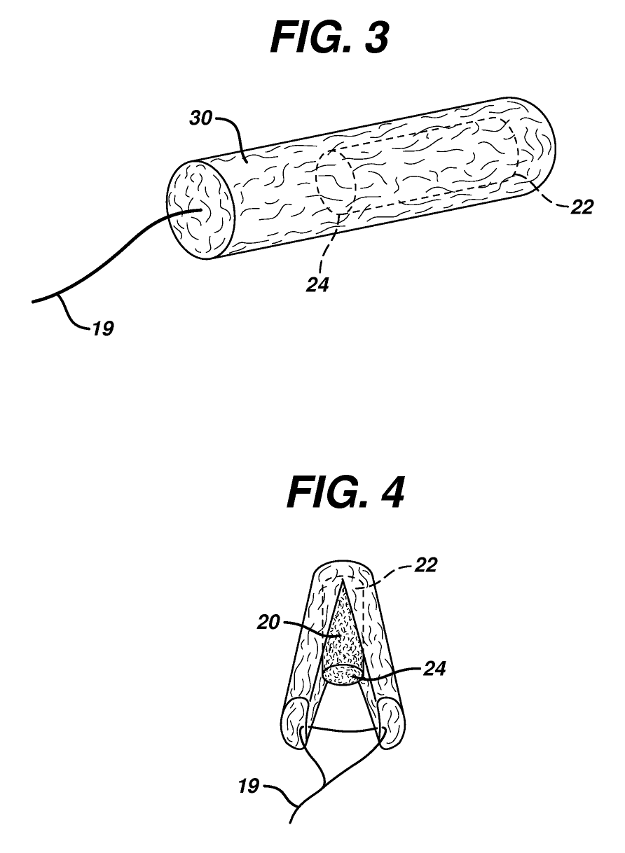

An alternative embodiment of the present invention is shown in FIGS. 3 and 4. The tampon 10 is again formed of a central, compressed elongate primary absorbent structure 20 and a secondary absorbent structure 30 extending from a first end 22 and toward a second end 24 of the primary absorbent structure 20. In use, the tampon 10 is inserted into a body cavity with the first end 22, corresponding to the insertion end 14 of the tampon 10, directed toward a source of bodily fluid discharges. Early fluid insults of the tampon 10 should result in the primary absorbent structure absorbing 20 sufficient fluid to expand radially, forcing the secondary absorbent structure 30 to extend outwardly to increase the cross-sectional area of the tampon 10, as shown in FIG. 4.

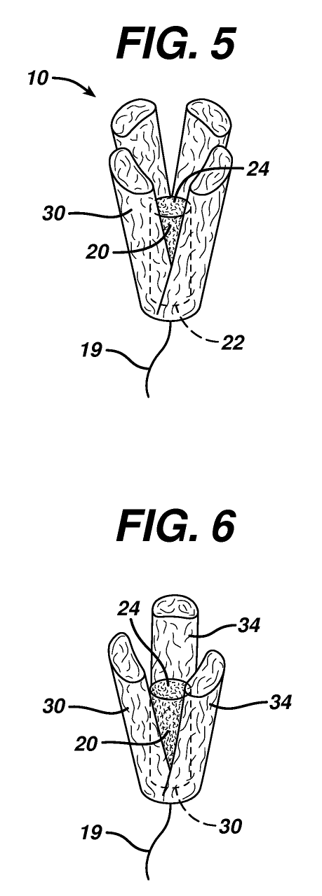

While the secondary absorbent structure 30 in the embodiments of FIGS. 1-4 is shown with two lobes 34a and 34b, the secondary absorbent structure 30 can have any reasonable number of elements defining any reasonable number of lobes. Additional examples in an expanded state are illustrated in FIGS. 5 (four lobes formed, e.g., of two crossed elements) and 6 (three lobes).

Again, the primary absorbent structure is compressed to allow it to expand radially upon absorbing liquid. This can be achieved by forming the primary absorbent structure 20 of a compressed, relatively dense absorbent structure, while the secondary absorbent structure 30 is formed of relatively less densely compressed material. In this embodiment, it is preferred that the primary absorbent structure 20 has a density of at least about 0.3 g/cm³, and more preferably, it has a density of at least about 0.4 g/cm³ . In another embodiment, the primary absorbent structure 20 is formed of a more resilient material than the secondary absorbent structure 30, while the respective densities are similar.

In the embodiment of FIGS. 1 and 2, the primary absorbent structure 20 and secondary absorbent structure 30 are compressed fibrous structures, each of which is substantially enclosed in a cover 18. An example of such an embodiment is formed by manufacturing an uncompressed tampon blank suitable to form a tampon of the mini absorbency level (6 to 9 ml) and securing the attachment zone 32 comprising the central portion of an elongate secondary absorbent structure 30 formed of additional absorbent sliver material to the first end 22 of the primary absorbent structure 20. The primary absorbent structure 20 can be further compressed or left unprocessed prior to the attachment of the secondary absorbent structure 30. The secondary absorbent structure 30 itself can be appended to the mini absorbency tampon as compressed or as an uncompressed blank. The added absorbency of the secondary absorbent structure 30 can then boost the absorbency of the complete tampon structure to a level of regular absorbency (9 to 12 ml). The tampon structure is then compressed to provide a tampon 10 having a higher density primary absorbent structure 20 and a lower density secondary absorbent structure 30.

In another embodiment, the primary absorbent structure 20 and secondary absorbent structure 30 are formed much as described above. However, the primary absorbent structure 20 has a greater percentage of highly resilient fibers than the secondary absorbent structure 30. When compressed, the primary absorbent structure 20 and secondary absorbent structure 30 have similar density, but the primary absorbent structure 20 is capable of expanding to a greater extent due to its greater percentage of highly resilient fibers.

Absorbent materials useful in the present invention include natural cellulose materials such as cotton, wood pulp, jute, hemp, sphagnum, and the like; and processed cellulose materials including cellulose derivatives such as regenerated cellulose (including rayon and lyocell), cellulose nitrate, carboxymethyl cellulose, and the like. Other useful materials include, without limitation, polyester, polyvinyl alcohol, polyolefin, polyamine, polyamide, polyacrylonitrile, and the like. Absorbent materials may be in the form of fiber, foam, superabsorbents, hydrogels, and the like. Preferred forms include fiber and foam.

Preferably, the materials include hydrophilic fibers, and more preferably, the fibers include absorbent fibers, i.e., the individual fibers, themselves, absorb fluid. Preferred foams, include hydrophilic foams, i.e., the foam cells, themselves, absorb fluid. Other materials, in addition to the above materials, may be included to add desirable characteristics to the absorbent body. For example, hydrophobic fibers may be used in outer surfaces of the tampon to reduce surface wetness and hydrophilic fibers may be used to increase the rate of fluid transport into and throughout the body.

The fibers used may have any useful cross-section.

For example, fiber cross-sections may be multi-limbed or non-limbed. In one embodiment, the fibers are predominantly multi-limbed. Multi-limbed, regenerated cellulosic fibers have been commercially available for a number of years. These fibers are known to possess increased specific absorbency over non-limbed fibers. One commercial example of these fibers are the Danufil VY multilimbed viscose rayon fibers available from Acordis Ltd., Spondon, England. These fibers are described in detail in Wilkes et al., U.S. Pat. No. 5,458,835, the disclosure of which is hereby incorporated by reference.

The primary and secondary absorbent structures 20, 30 are preferably substantially enclosed by a fluid-permeable cover 18. Thus, the cover 18 encloses a majority of the outer surface of the tampon. This may be achieved as disclosed in Friese, U.S. Pat. No. 4,816,100, the disclosure of which is herein incorporated by reference. In addition, either or both ends of the primary and secondary absorbent structures 20, 30 may be enclosed by the cover 18. Of course, for processing or other reasons, some portions of the surface of the tampon 10 may be free of the cover 18.

The cover 18 can ease the insertion of the tampon 10 into the body cavity and can reduce the possibility of fibers being separated from the tampon 10. Useful covers are known to those of ordinary skill in the art.

They may be selected from an outer layer of fibers which are fused together (such as by thermobonding), a nonwoven fabric, an apertured film, or the like. In one embodiment, the cover has a hydrophobic finish to minimize over-drying of body cavity tissue.

Tampons are generally categorized in two classes: applicator tampons and digital tampons. Applicator tampons use a relatively rigid device to contain and protect the tampon prior to use. To insert the tampon into a body cavity, the applicator is partially inserted into the body cavity, and the tampon can be expelled therefrom. Because the rigid applicator device protects the tampon, the tampon need not have a high degree of dimensional stability. In contrast, digital tampons do not have an applicator to help guide them into the body cavity and require sufficient stability to allow insertion without using an applicator.

When pressure is released after moderate mechanical compression, a tampon tends to expand toward its original dimensions. Therefore, tampon blanks are generally over-compressed during manufacture to allow them to rebound slightly to the desired density for use.

Over-compression mechanically constricts expansion to prevent the tampon from expanding without added liquid.

The over-compression of the mass of the absorbent material which form the tampon provides some degree of dimensional stability, especially longitudinal crush resistance. This measurement can be described as the tampon's column strength. This strength can be determined by securing one end of the tampon to the fixed plate of a Instron Universal Testing Machine, available from Instron Corporation, Canton, Mass., USA. The moveable plate is brought to contact the opposite end of the tampon and is then set to compress the tampon at a rate of about 5 cm/minute. The force exerted on the tampon is measured continuously, and the point at which this force begins to fall instead of rise is the point at which the tampon buckles. The maximum force achieved is the tampon column strength. Preferably, digital tampons of the present invention have significant column strength, at least about 10 Newtons (N). More preferably, the digital tampons have a column strength of at least about 20 N, and most preferably, they have a column strength of about 30 N to about 85 N. Tampons with a column strength that is too low do not have sufficient dimensional stability to maintain their basic structure during insertion as a digital tampon; tampons with a column strength which is too high can be perceived as being too stiff or hard to be comfortably inserted as a digital tampon.

During use, the primary absorbent structure 20 of the present tampon 10 absorbs moisture and liquids and radially expands. The primary absorbent structure 20 expands in at least one direction perpendicular to the central axis, preferably, in at least two directions. Most preferably, the primary absorbent structure 20 of the tampons expand substantially uniformly in all directions perpendicular to the central axis “A”. This radial expansion of the primary absorbent structure 20 provides the mechanical force to move the secondary absorbent structure 30 outwardly into an extended position. This outward movement of the secondary absorbent structure 30 provides a greater cross-sectional area during use of the tampon in a user's body.

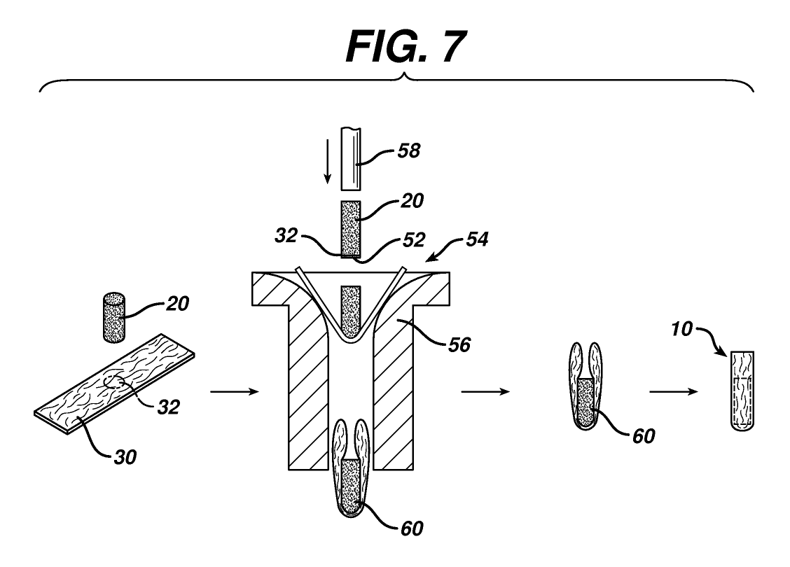

Referring to FIG. 7, the primary absorbent structure 20 can be formed of a compressed rod, such as a tampon structure of mini absorbency (6 to 9 ml). An attachment zone 32 comprising the central portion of an elongate secondary absorbent structure 30 formed of additional absorbent sliver material can be secured to the first end 22 of the primary absorbent structure 20.

The primary absorbent structure 20 can be formed as is known to those of ordinary skill in the art. One example of this is as a mini absorbency tampon, such as disclosed in Friese et al., U.S. Pat. No. 6,310,269, the disclosure of which is herein incorporated by reference.

The secondary absorbent structure 30 can be appended to the primary absorbency structure 20 when the primary absorbent structure 20 is pre-compressed or is an uncompressed blank. Preferably, the primary absorbent structure 20 is pre-compressed. The secondary absorbent structure 30 can be secured to the primary absorbent structure 20 by adhesive 52, heat sealing, threading the withdrawal string 19 through both structures, and the like, to form an assembly 54. The assembly 54 can then be driven through a conical bushing 56 with a rod 58 to form a generally cylindrical tampon blank 60. The tampon blank 60 can then be finished by compressing it in a tampon press (not shown, e.g., as disclosed in Friese et al., U.S. Pat. No. 6,310,269), to form the tampon 10, and then packaged.

### EXAMPLES

The present invention will be further understood by reference to the following specific Example that is illustrative of the composition, form and method of producing the device of the present invention. It is to be understood that many variations of composition, form and method of producing the device would be apparent to those skilled in the art. The following Example, wherein parts and percentages are by weight unless otherwise indicated, is only illustrative.

A pre-compressed, cylindrical rod having a diameter of about 11 mm and a length of about 30 mm which was derived from a tampon of the mini absorbency level (6 to 9 ml) enclosed in a nonwoven cover as generally described in Friese, U.S. Pat. No. 4,816,100 was used as the primary absorbent structure. The center portion of a secondary absorbent structure formed of a rectangular fibrous web enclosed in a similar nonwoven cover having a width of about 30 mm and a length of about 130 mm was adhered to one end of the primary absorbent structure. Both absorbent structures were formed of a blend of 75 wt-% Danufil VY multilimbed viscose rayon fibers and 25 wt-% Danufil V viscose rayon fibers.

A string was looped through the primary and secondary absorbent structures, and the free ends of the secondary absorbent structure was folded to substantially enclose the primary absorbent structure. The resulting construction was compressed as described in Friese et al., U.S. Pat. No. 6,310,269, to form a compressed tampon of regular absorbency.

A control group of regular absorbency tampons was manufactured as generally described in Friese et al., U.S. Pat. No. 6,310,269. The column strength of the resulting tampon was measured as described above using an Instron Universal Testing Machine. The weight of the tampons and the column strength, in Newtons (N), are reported below.

| | |
|---|---|
| Example 1 | Control |

| | Column | | Column |
|---|---|---|---|
| W | Strength | W | Strength |
| (g) | (N) | (g) | (N) |
| 2.85 | 75.1 | 2.89 | 69.2 |
| 2.86 | 75.6 | 2.58 | 49.6 |
| 2.88 | 75.3 | 2.72 | 64.6 |
| 2.84 | 76.0 | 2.63 | 53.0 |
| 2.81 | 75.7 | 2.74 | 71.3 |
| 2.82 | 75.0 | 2.66 | 63.6 |
| 2.81 | 75.1 | 2.80 | 58.2 |
| 2.84 | 76.3 | 2.86 | 61.9 |
| 2.80 | 75.1 | 2.65 | 57.9 |
| 2.85 | 75.8 | 2.66 | 60.7 |

The data reveal that the tampons of Example 1 exhibit a column strength at least as great as the control tampons.

The specification and embodiments above are presented to aid in the complete and non-limiting understanding of the invention disclosed herein. Since many variations and embodiments of the invention can be made without departing from its spirit and scope, the invention resides in the claims hereinafter appended.

1-13. (canceled)

14. An absorbent tampon suitable for insertion into a body cavity comprising:
a) a substantially elongate, compressed primary absorbent structure having a longitudinal axis, a first end, and a second end disposed opposite thereof; the primary absorbent structure being capable of expanding upon exposure to the bodily fluid discharge to increase a radius, perpendicular to the longitudinal axis by at least about 40%; and
b) a secondary absorbent structure secured to and extending from the first end of the primary absorbent structure and extending toward the second end of the primary absorbent structure;
wherein the expansion of the primary absorbent structure opens the secondary absorbent structure to increase the cross-sectional area.

15. A digital, absorbent, vaginal tampon comprising:
a) a substantially elongate, compressed primary absorbent structure having a longitudinal axis, a first end, and a second end disposed opposite thereof; the primary absorbent structure being capable of expanding upon exposure to the bodily fluid discharge to increase a radius, perpendicular to the longitudinal axis by at least about 40%; and
b) a secondary absorbent structure secured to and extending from the first end of the primary absorbent structure and extending toward the second end of the primary absorbent structure;
wherein the tampon has a column strength of at least about 10 N prior to expansion, and the expansion of the primary absorbent structure opens the secondary absorbent structure to increase the cross-sectional area.

16. A process for making an absorbent tampon suitable for insertion into a body cavity, the process comprising the steps of:
a) forming a substantially cylindrical, radially expanding primary absorbent structure having a first end and a second end disposed opposite thereof;
b) securing an attachment zone of a secondary absorbent structure to the first end of the primary absorbent structure to form a tampon assembly;
c) directing at least one distal portion of the secondary absorbent structure toward the first end of the primary absorbent structure, wherein the at least one distal portion is located at an edge of the secondary absorbent structure and spaced distal the attachment zone; and
d) compressing the tampon assembly to form the absorbent tampon;
wherein the absorbent tampon has a column strength of at least about 10 N.

17. The process of claim 16 wherein the step of securing the attachment zone of the secondary absorbent structure to the first end of the primary absorbent structure comprises applying an adhesive therebetween.

18. The process of claim 16 wherein the step of securing the attachment zone of the secondary absorbent structure to the first end of the primary absorbent structure comprises heating either the attachment zone or the first end of the primary absorbent structure.

19. The process of claim 16 wherein the step of securing the attachment zone of the secondary absorbent structure to the first end of the primary absorbent structure comprises passing a string through the attachment zone and the first end of the primary absorbent structure.

20. The process of claim 16 wherein the step of directing at least one distal portion of the secondary absorbent structure toward the first end of the primary absorbent structure comprises passing the tampon assembly through a conical bushing.
